(19) United States
(12) Patent Application Publication
(10) Pub. No.: US 2004/0037773 A1
(43) Pub. Date: Feb. 26, 2004

(54) Method for detecting abnormal tissue using enhanced radiopharmaceutical uptake

(75) Inventor: Richard M. Fleming, Omaha, NE (US)

Correspondence Address: FOLEY & LARDNER, 321 NORTH CLARK STREET, SUITE 2800, CHICAGO, IL, 60610-4764, US

(73) Assignee: Block Medical Center

(21) Appl. No.: 10/603,841

(22) Filed: Jun. 25, 2003

Related U.S. Application Data
(60) Provisional application No. 60/391,708, filed on Jun. 26, 2002.

(51) Int. Cl. A61K 51/00; A61K 31/519

(52) U.S. Cl. 424/1.11; 514/262.1

(57) ABSTRACT

The invention provides a method for detecting abnormal tissue by enhancing the vascularity of abnormal tissue using a vasodilatory agent prior to administering a radiopharmaceutical which selectively accumulates in abnormal tissue. In one embodiment the vasodilatory agent is dipyridamole and the radiopharmaceutical is is Technetium-99m hexakis 2-methoxyisobutylisonitrile (sestamibi).

### FIELD OF THE INVENTION

The present invention relates to methods for detecting abnormal tissue using vasodilitory agents to enhance radiopharmaceutical uptake in tissues.

### BACKGROUND OF THE INVENTION

Technetium-99m hexakis 2-methoxyisobutylisonitrile (sestamibi) is an isotope that emits low levels of gamma radiation. In the body, sestamibi gets picked up by cells, where it can be detected using physiologic imaging techniques designed to measure the radiation emitted by the cells. Sestamibi is preferentially taken up by certain abnormal cells, such as those found in cancerous tissues, and cancerous breast tissues in particular.

Sestamibi imaging (such as Miraluma®, a imaging test marketed by Bristol-Myers Squibb Medical Imaging, Inc., a subsidiary of Bristol-Myers Squibb, Inc. with headquarters at 345 Park Ave New York N.Y. 10154) is used to enhance the detection of breast cancers and is a useful adjunct to mammography. The delivery and subsequent detection of sestamibi uptake by a tumor is dependent upon (a) the delivery of the isotope to the tumor through blood flow to the tumor and (b) the presence of living tissue with active mitochondria. It has been shown that over about 90% of sestamibi is taken up by mitochondria in an energy dependent manner. This uptake increases with the number of mitochondria present. Also, this uptake increases with the functional activity of the mitochondria, that is, the greater the activity of the cells, the greater the uptake of the mitochondria.

In addition to mitochondrial activity, the presence of sestamibi is dependent upon its delivery through the bloodstream to the region of the body being imaged. Regions of abnormality, such as inflammation, atypia and cancers, which produce angiogenic factors, have greater vascularity than do normal tissues which increase the delivery of sestamibi to tissues for imaging.

Inflammatory cells typically take up sestamibi to a greater extent than normal cells, but to a lesser degree than cancer cells. This uptake demonstrates the ability of the cells to maintain a transmembrane potential. Unfortunately, the contrast in the sestamibi uptake between normal and abnormal tissues can be insufficient to make accurate diagnosis regarding the present of abnormal tissues. For this reason, the use of sestamibi imaging technology for detecting abnormal cells, such as cancer, has been limited. Thus a need exists for a method of detecting abnormal cells and tissues using sestamibi imaging with an enhanced contrast between abnormal and normal cells.

Like breast cancer, the detection of coronary artery disease also may be determined by physiologic (Single-Photon Computed Tomography (SPECT) or Positron Emission Tomography (PET) imaging) methods. The ability to change coronary blood flow using high-dose dipyridamole (HDD) to detect coronary artery disease has been previously demonstrated. Enhancement of blood flow to the heart using HDD has proven useful in unmasking heart disease through the augmentation of regional blood flow differences. Specifically, HDD has been administered to patients to enhance cardiac imaging to demonstrate ischemic, infarcted and viable myocardium, as well as to determine doxorubicin (marketed under the trade name Adriamycin®) induced cardiotoxicity following chemotherapy. Until now, however, the advantages of combining sestamibi imaging with the vasodilatory effects of HDD have not been realized in the areas of inflammatory tissue and cancer diagnosis.

### SUMMARY OF THE INVENTION

The present invention provides a method for the early detection of abnormal tissue. The present invention effectuates early detection of abnormal tissue using radiopharmaceutical imaging by increasing the vascularity of abnormal tissue, thereby enhancing mitochondrial activity and increasing the delivery of the radiopharmaceutical to abnormal tissues. Briefly, the method of the present invention includes administering to a patient a vasodilatory agent that is capable of increasing the uptake of a radiopharmaceutical which selectively accumulates in abnormal tissues. The administration of the vasodilatory agent is followed by the administration of the radiopharmaceutical. Finally, the patient's tissue of interest is imaged with a radiation detector to detect the abnormal tissue in the patient.

In one embodiment the present invention a vasodilatory agent is used to detect abnormal breast tissue in a patient. In this embodiment dipyridamole (HDD) is a preferred vasodilatory agent and sesamibi is a preferred radiopharmaceutical.

### BRIEF DESCRIPTION OF THE DRAWINGS

FIG. 1 shows resting (Miraluma®) and Breast Enhanced Scintigraphy Test (B.E.S.T.) imaging protocols.

FIG. 2a shows examples of Miraluma® and B.E.S.T. images in the same patient.

FIG. 2b shows the sequence of processing images required to derive the final blue-green image and Maximal Count Activity (MCA) display of a B.E.S.T. image.

FIG. 3 shows a comparison of MCA obtained using Miraluma® and B.E.S.T. imaging.

FIG. 4 shows a graphic representation of the results of MCA as seen in normal breast tissue, inflammatory tissue and breast cancer tissue, obtained using B.E.S.T. imaging.

FIG. 5 shows differences in MCA individuals with normal, inflammatory, atypia and cancerous breast tissue.

### DETAILED DESCRIPTION OF A PREFERRED EMBODIMENT

The present invention provides an improved method for the early detection of abnormal tissues. In one method of the present invention, the uptake of a radiopharmaceutical by abnormal tissues is increased by administering to a patient a vasodilitory agent prior to the administration of the radiopharmaceutical. This results in an increase in the radiation emitted from the tissue and, subsequently, a clearer image of abnormal tissues as detected by physiological techniques. In one embodiment of the present invention, the uptake of the radiopharmaceutical by abnormal tissues following administration of a vasodilitory agent is increased exponentially compared to the uptake of the radioisotope by normal tissues.

When used to detect abnormal breast tissues, the method of the present invention will be referred to as “Breast Enhanced Scintigraphy Testing” or “B.E.S.T.” A description of the B.E.S.T. imaging method may be found in Fleming R M, Dooley W C, Boyd L B, Kubovy C, “Breast Enhanced Scintigraphy Testing (B.E.S.T.)—Increased accuracy in detecting breast cancer accomplished by combining breast and cardiac imaging,” 48th Annual Scientific Session of the Society of Nuclear Medicine, Toronto, Ontario, Canada, 26 Jun. 2001, the disclosure of which is incorporated herein by this reference.

The uptake of radiopharmaceuticals by cells in the body is dependent upon both the delivery of the radioactive isotope through the vascular bed and the presence of mitochondrial activity once the isotope has been delivered. Without intending to be bound to any particular theory of the invention, the inventors believe that the increase in isotope uptake in the method of this invention is due to a combination of increased vascularity in the abnormal tissues and increased mitochondrial activity present in leukocytes when compared to normal tissue. Cancerous tissues theoretically have increased vascularity resulting from angiogenic activity, which increases blood supply to the cancer providing nutrients for growth and survival. In addition, cancers and inflammatory tissue have greater mitochondrial activity than normal tissue, which has greater activity than injured or necrotic tissue.

The basic steps of the method according to the present invention include: .1) administering a vasodilatory agent to a patient; 2) administering a radiopharmaceutical which selectively accumulates in abnormal tissue to the patient; and 3) imaging the tissue of the patient with a radiation detector to detect the abnormal tissue in the patient. The invention may be used to increase the radiopharmaceutical uptake in a variety of abnormal tissues. Abnormal tissues are tissues other than normal healthy tissues and may include inflammatory tissue, atypia tissue, and cancerous tissue. When used herein, the term “atypia” means a deviation from normal or the typical. When used herein, the term “inflammatory” means a state of tissue response to injury. When used herein, the term “cancer” means a state of tissue of potentially unlimited growth that expands locally by invasion and systemically by metastasis

The vasodilatory agent may be any agent capable of increasing blood flow to abnormal tissues. A biologically effective amount of the vasodilatory agent is an amount that is sufficient to induce increased blood flow in abnormal tissues. Dipyridamole (HDD) is a vasodilatory agent that is particularly suited for enhancing the detection of abnormal tissues according to the present invention. In an alternative version in accordance with the principles of the present invention, adenosine and nitroglycerin could be utilized as vasodilatory agents. In addition, dobutamine has shown promise in pharmacological imaging of the heart and may be a useful marker. See Fleming R M, Feldmann K M, and Fleming D M, “Comparing a High Dose Diyridamole SPECT Imaging protocol with Dobutamine and Exercise Stress Testing Protocols. Part III: Using Dobutamine to Determine Lung-to-Heart Ratios, Left Ventricular Dysfunction and a potential Viability Marker,” Intern J of Angiol 1999, 8:22-26, the disclosure of which is incorporated herein by reference.

Radiopharmaceuticals are radioactive compounds or drugs that contain one or more radioactive isotopes. Radiopharmaceuticals are taken up by cells in the body from which they emit radiation (gamma rays). A biologically effective amount of a radiopharmaceutical is an amount that is sufficient to provide a detectable level of radiation when taken up by the tissue of interest in the body. Radiopharmaceuticals for use with the present invention may be any radiopharmaceutical capable of selectively accumulating in at least one type of tissue. One such radiopharmaceutical is sestamibi which selectively accumulates in abnormal breast tissues. In an alternative version in accordance with the principles of the present invention, thallium-201, technetium isotopes such as but not limited to myoview, 18-flurodeoxyglucose (FDG) and fatty acid analogue could be utilized as radiopharmaceutical agents.

The radiation detector may be any detection system capable of detecting the radiation emanating from the pharmaceutical within the patient's body and imaging the abnormal tissue from which the radiation originates. Such radiation detectors are well known and include, but are not limited to, single photon emission computed tomography (SPECT) detectors, positron emission tomography (PET) detectors, semiconductor detectors, other suitable planar imaging devices, and any other radiation detection device to be developed.

If desired, the imaging of the tissue of interest using the method of the present invention may be followed by cardiac imaging techniques utilizing the same radiopharmaceutical. Such cardiac imaging techniques are well-known. Examples of such techniques are described in Fleming R M, “Chapter 29 of Atherosclerosis: Understanding the Relationship Between Coronary Artery Disease and Stenosis Flow Reserve,” Textbook of Angiology, John C. Chang Editor, Springer-Verlag New York, N.Y. 1999, pp. 381-387; Fleming R M, “Chapter 31. Nuclear Cardiology: Its Role in the Detection and Management of Coronary Artery Disease,” Textbook of Angiology, John C. Chang Editor, Springer-Verlag New York, N.Y. 1999, pp. 397-406; Fleming R M, Boyd L, Forster M, “Angina is Caused by Regional Blood Flow Differences—Proof of a Physiologic (Not Anatomic) Narrowing,” Joint Session of the European Society-American College of Cardiology, ACC 49th Annual Scientific Sessions, Mar. 12, 2000 (www.prous.com); Fleming R M, “Regional Blood Flow Differences Induced by High Dose Dipyridamole Explain Etiology of Angina,” 3rd International College of Coronary Artery Disease from Prevention to Intervention,” Lyon, France, Oct. 4, 2000; Fleming R M, Boyd L B, Kubovy C, “Myocardial perfusion imaging using high dose dipyridamole defines angina. The difference between coronary artery disease (CAD) and coronary lumen disease (CLD),” 48th Annual Scientific Session of the Society of Nuclear Medicine, Toronto, Ontario, Canada, 27 Jun. 2001; Fleming R M, “Coronary artery disease is more than just coronary lumen disease,” Am J Card 2001, 88:599-600; the disclosures of which are incorporated herein by reference.

A brief exemplary description of the B.E.S.T. method of the present invention follows, a more detailed description of specific embodiments are illustrated in the examples below. The patient may be prepared for B.E.S.T. imaging by placing the patient in a prone position with the breast to be imaged supported with inserts which position the breast for imaging. Once the patient is suitably comfortable, a biologically effective amount of the vasodilatory agent is administered, preferably intravenously. If the vasodilatory agent is HDD, a biologically effective amount will typically be between about 30 mg and about 120 mg

After sufficient time has passed to allow the vasodilatory effects of the vasodilatory agent to take effect, typically about 3 to about 5 minutes, a biologically effective amount of a radiopharmaceutical is administered to the patient. The radiopharmaceutical is preferably administered intravenously by injecting it into the arm or breast contralateral to the breast to be imaged. If the radiopharmaceutical is sestamibi, a biologically effective amount will typically be between about 20 and about 35 mCi.

Imaging of the breast should commence once the radiopharmaceutical has been in the patient's system long enough to allow for substantial uptake of the radiopharmaceutical by abnormal tissues in the breast. Typically, imaging will begin approximately 10 minutes after the onset of the administration of the vasodilatory agent. The breast tissue may be imaged by any suitable scintillation detector, including a SPECT or PET camera. If the imaging is done by a SPECT camera, abnormal tissues will appear as areas of maximal count activity (MCA) in the images. These areas represent the region of breast tissue with the greatest blood flow and mitochondrial activity. MCA is a measure of inflammatory breast tissue. The analysis of MCA may be performed using computer assessment and areas of the MCA may be displayed on a computer monitor. In a preferred embodiment of the invention, the MCA for abnormal tissues is increased without substantially altering the MCA of normal tissues, resulting in a more pronounced imaging contrast between normal and abnormal tissues.

Enhancement of the delivery of sestamibi to breast tissue according to the present invention not only allows for the distinction of “normal” tissue versus “breast cancer” tissue, but a distinction between “normal” regions of “inflammatory” changes of the breast, regions of cellular “atypia” tissue and breast “cancer” tissue. There is logically a progression from “normal” breast tissue to breast “cancer” tissue. While not all regions of inflammatory changes are destined to become cancer, these regions may represent regions at greater risk of becoming a cancer, subsequently needing closer monitoring to determine if they are progressing to the development of a cancer. Clearly, the sooner a cancer is detected, the greater is the likelihood of successful treatment.

The appearance of lesions seen on B.E.S.T. imaging also may be used to distinguish differences in the appearance of cancers and pre-cancers. Pre-cancers and cancers do not look alike when detected by physiologic methods. For example, ductal carcinoma in situ (DCIS) appear tubular, following the path of the milk ducts, while breast cancer appears spherical. However, once these cells have changed further, they no longer obey contact inhibition and subsequently grow into surrounding tissue producing a spherical appearance in the same way they behave in the laboratory. These changes in appearance therefore can be used to distinguish early/pre-cancers from the next stage of infiltrating cancer. By enhancing the images and the contrast between normal and abnormal tissues, the present invention makes it easier to distinguish between abnormal tissue samples having various shapes and sizes.

The invention is described in greater detail in the following non-limiting examples. While the present invention has been tested and described in connection with the detection of breast cancer, the principles of the present invention are equally applicable to so-called “hard tumors” (pre-cancerous and cancerous) or other origins or locations. Thus, it is neither intended nor should the present invention be interpreted as being limited solely to the detection of breast cancer.

### EXAMPLES:

In the examples that follow, two hundred and five (205) individuals who ranged in age from 27 to 88 years (51±11 years) of age were studied during a thirty three (33) month period beginning in February 1999 and ending in November 2001. The group included 201 women and 4 men. The individuals included 181 Caucasians, 5 Hispanics, 17 African American and 2 people of Mediterranean origin. No differences in outcomes were found based upon age, race or sex. Women were excluded from the study if they were taking hormone replacement therapy, were pregnant or were breast-feeding. All subjects signed consent forms prior to undergoing breast imaging.

Histopathologic information was obtained for all but 58 of the individuals studied. Tissue samples were obtained either through ductoscopy, fine needle aspiration or open biopsy and were interpreted by pathologists without knowledge of the sestamibi image results. Results were interpreted as being normal, inflammatory (evidence of leukocytes), atypia (with increasing order of cellular change progressing from hyperplasia to metaplasia/atypia to ductal carcinoma in situ) and cancer.

Two sestamibi imaging studies were conducted. The first study was conducted to demonstrate that breast cancer has increased vascularity which can be influenced by the vasodilatory effect of dipyridamole (HDD), and greater mitochondrial activity the detection of which may be enhanced once increased delivery of sestamibi is provided through enhanced blood flow. In this study, breast tissues of ten women were studied using a conventional resting sestamibi imaging (Miraluma®) approach and these results were compared with results obtained following enhanced delivery of the isotope using HDD (i.e. B.E.S.T. imaging). The results were also compared with biopsy data. All 10 women in the study had either an abnormality on mammography or a detectable lump on physical examination.

In a second study, one hundred and ninety five (195) (4 men, 191 women) individuals were studied using the enhanced (HDD) imaging approach. The study included four men having detectable breast lumps and 191 women, including 58 seeking additional information regarding breast disease concerns, and 133 with breast lumps and/or abnormal mammograms. The results of this study were also compared with biopsy data.

### Resting Sestamibi Breast Imaging (Miraluma®)

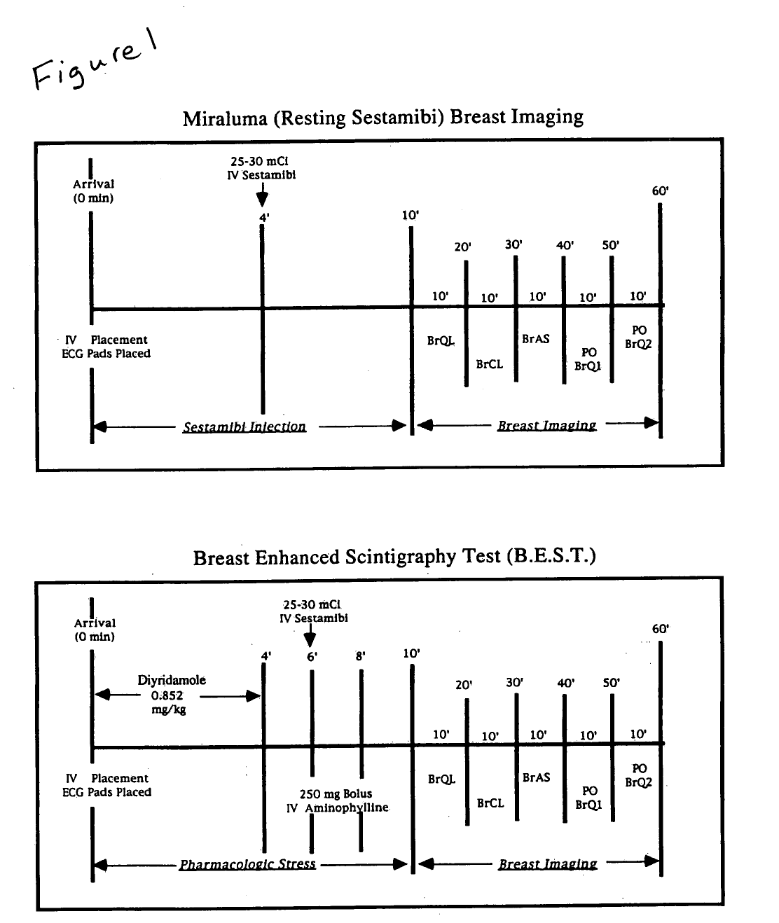

Subjects undergoing breast imaging arrived in the fasting state 15-30 minutes prior to the study. FIG. 1 shows Miraluma® and Breast Enhanced Scintigraphy Test (B.E.S.T.) imaging protocols. The top panel displays the protocol for resting sestamibi heart imaging of the breast using the isotope sestamibi. Subjects had a 20 gauge intravenous catheter placed either in the right antebrachium or the left antebrachium if there is a specific question regarding the right breast. Sestamibi was administered intravenously four minutes into the study followed by a 10-20 cc normal saline flush to assure delivery of the isotope into the venous system, with imaging of the breast beginning ten minutes into the study. A SPECT camera was positioned in a stationary (planar) position for each of the images.

Breast imaging began with the patient placed in a prone position on top of a 6-inch foam pad designed to enhance the comfort of the patient while improving breast imaging. Each side of the 6-inch pad had breast inserts held in place by hook and loop-type adhesive strips, which were removed allowing each breast to be positioned through the openings in a dependent manner without breast compression. Planar breast imaging began with the BrQL breast (lateral view of breast in question or the right breast if neither breast was specifically suspected of having an abnormality), then the BrCL breast (lateral view of the breast contralateral to BrQL). Patients were then placed on their back for the anterior (BrAS) image of both breasts. Any areas of special concern, PO BrQ1 (posterior oblique view of 1st breast noted to have abnormal activity on initial views) and PO BrQ2 (posterior oblique view of the other breast if its activity is abnormal) were then imaged.

As shown in FIG. 1, 25-30 mCi (925-1110 MBq) of Technetium-99m hexakis 2-methoxyisobutylisonitrile (sestamibi) was administered intravenously at the 4-minute mark with image acquisition beginning 6 minutes later. Ten minutes into the study, image acquisition was started, as shown in FIG. 1. All images were acquired while the patient was prone, except for the anterior (BrAS) image, which was obtained while the patient was supine.

Breast image acquisition and reconstruction was performed using a Siemens orbiter Single Photon Emission Computed Tomography (SPECT) camera with 75 photomultiplier tubes (PMTs) and a 128 by 128 matrix, available from Siemens Medical Solutions, Malvern, Pa. The images were acquired with the camera head in a stationary (planar) position. The camera, computer and software providing quantification of maximal count activity (MCA) were supplied by NC Systems of Boulder, Colo. A low-energy high-resolution (LEHR) collimator was used providing a resolution of 3.4 mm.

Cardiac imaging could have been initiated immediately after completion of the breast imaging, if desired.

### Breast Enhanced Scintigraphy Test (B.E.S.T.) Imaging

The bottom panel of FIG. 1 displays the protocol for Breast Enhanced Scintigraphy Testing (B.E.S.T.) imaging.

Subjects undergoing B.E.S.T. imaging were prepared for the study in a manner identical to that used in preparation for the Miraluma® imaging. Subjects arrive in a fasting state 15-30 minutes prior to the study. A 20 gauge intravenous catheter was placed either in the right arm, or in the left arm if the right breast was the breast in question. As shown in FIG. 1, enhancement of blood flow was provided by administering 0.852 milligram per kilogram (mg/kg) body weight of intravenous dipyridamole (HDD) through an intravenous catheter, infused over 4 minutes. The catheter was flushed with 10-20 cc of normal saline immediately after the HDD had been given to assure introduction of all of the HDD into the venous system. Two minutes later, at peak dipyridamole effect, 25-30 mCi (925-1110 MBq) of is Technetium-99m hexakis 2-methoxyisobutylisonitrile (sestamibi) was administered intravenously. Image acquisition was started 10 minutes into the study as shown in FIG. 1, with the patient in a prone position. Anterior images were obtained with the patient in a supine position.

Following breast imaging, cardiac imaging was performed as described previously, providing information regarding coronary blood flow, regional wall abnormalities and left ventricular ejection fraction, all of which are useful for making further diagnostic decisions, particularly regarding the use of chemotherapy and radiation therapy. Cardiac Imaging is performed using gated images beginning immediately after completion of the breast imaging using a Siemens orbiter Single Photon Emission Computed Tomography (SPECT) camera with 75 photomultiplier tubes (PMTs) and a 128 by 128 matrix, available from Siemens Medical Solutions, Malvern, Pa. The images were acquired with the camera head in a stationary (planar) position. The camera, computer and software providing quantification of maximal count activity (MCA) were supplied by NC Systems of Boulder, Colo. A low-energy high-resolution (LEHR) collimator was used providing a resolution of 3.4 mm. Image acquisition required about 32 minutes using a step and shoot approach. For a description of other cardiac imaging techniques see Fleming R M, Chapter 31, “Nuclear Cardiology: Its Role in the Detection and Management of Coronary Artery Disease,” Textbook of Angiology, pp. 397-406; Fleming R M, Rose C H, Feldmann K M, “Comparing a high-dose dipyridamole SPECT imaging protocol with dobutamine and exercise stress testing protocols,” Angiology 1995, 46:547-556, which are hereby incorporated by reference.

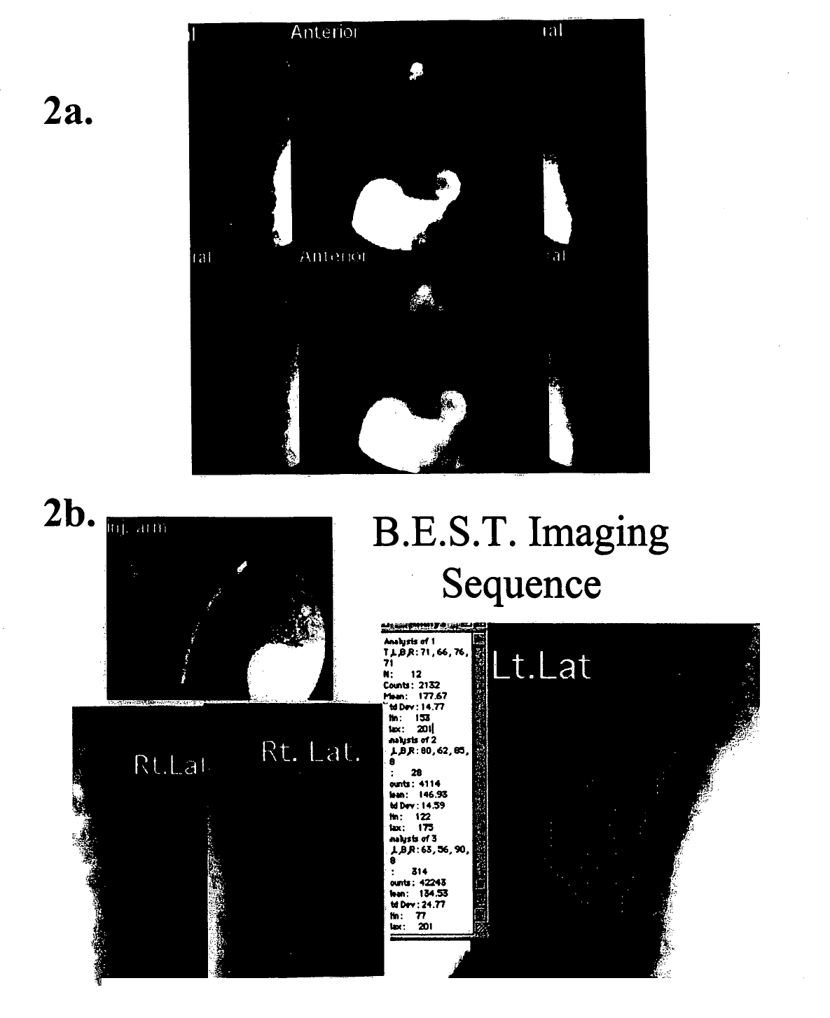

Breast imaging equipment and acquisition were identical to those used for the Miraluma® approach. The image display for B.E.S.T. was displayed in a blue-green format to reduce artifacts. An example of the blue-green format is shown in FIG. 2a, juxtaposed with resting images.

Following the image reconstruction and presentation of each breast image, the region of greatest maximal activity (MCA) was determined and quantified. MCA is a measure of detected radiation (gamma) emission acquired by the SPECT camera during the imaging process and reflects the amount of isotope taken up at any given time. See Fleming R M, Dooley W C, Boyd L B, Kubovy C, “Breast Enhanced Scintigraphy Testing (B.E.S.T.)—Increased accuracy in detecting breast cancer accomplished by combining breast and cardiac imaging,” 48th Annual Scientific Session of the Society of Nuclear Medicine, Toronto, Ontario, Canada, 26 Jun. 2001, the disclosure of which is incorporated herein by this reference. This assessment of MCA was performed using computer assessment of MCA as measured and displayed on the computer monitor. All readings and determination of MCA were determined for each breast prior to any knowledge of clinical, mammographic or pathologic information, which could in any way bias the results. These procedures were performed at a recognized Center of Excellence for Nuclear Procedures under the direct supervision of a physician Boarded in Nuclear Imaging.

FIG. 2b shows the sequence of processing images required to derive the final blue-green image and MCA display of a B.E.S.T. image. Several images are displayed showing the sequence of images obtained and processed to develop the final B.E.S.T. image with MCA measurement. The upper left image reveals the isotope (sestamibi) immediately after injection into the venous system of the right arm (inj. arm). Following image acquisition, a black and white image is displayed (Rt. Lat.) which is then converted into a blue-green image. Following this display of both breasts, the MCA is determined for each breast. In the case shown in FIG. 2b, the left breast had the greatest activity. Three regions of interest (ROIs) were measured for maximal count activity (MCA) and are displayed. Region 1 represents a smaller ROI in the upper middle breast with a MCA of 201. This ROI surrounds a milk duct. The second ROI was immediately below the first and had a MCA of 175. The third ROI included the entire breast, incorporating both the first and second ROI. Consequently the MCA of the third ROI included region one which had the greatest MCA of 201.

### Comparison of the Data and Statistical Analysis

FIG. 2a shows examples of Miraluma® and B.E.S.T. images in the same patient. The top row of images shows (from left to right) a lateral view of the left breast (Lt. Lat), anterior (BrAS) view of both breasts, and a lateral view of the right breast (Rt. Lat). These black and white images represent the results seen with Miraluma® imaging. The bottom row of blue-green images represents the same patient following imaging with enhanced Scintigraphy (B.E.S.T.) imaging. The black and white Miraluma® image was initially visually interpreted as abnormal with the appearance of increased tracer uptake (white) in the region of the left nipple; however, the MCA revealed “normal” breast tissue using both approaches.

Images were then displayed in a black and white format as shown in FIG. 2a. ROIs are then drawn around the entire breast and analysis was made for the greatest amount of tracer uptake. This greatest activity was the maximal count activity (MCA) and represents the region of breast tissue with the greatest blood flow and mitochondrial activity.

The outcomes of histopathologic specimens were compared with the MCA derived from sestamibi imaging. Descriptive statistical analysis of the MCAs were determined including mean±standard deviation and confidence intervals (CI) for the mean. Group comparisons were made using two-tailed t-tests to determine statistical differences defined as p-values of ≦0.05. Graphic representation of the means is shown for comparison purposes as well as raw data comparison for the histopathologic categories.

### Study 1: Results

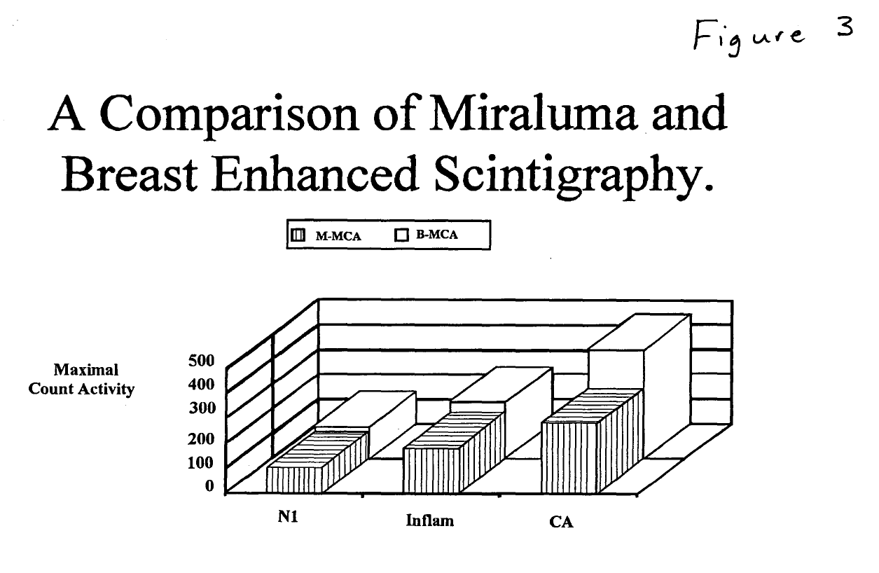

During the first part of the study ten women underwent biopsy in addition to both Miraluma® and B.E.S.T. imaging. Four of the women had normal breast tissue, four had inflammatory changes and two had breast cancer. FIG. 3 shows a comparison of MCA obtained using Miraluma® and B.E.S.T. imaging. This bar graph represents the mean MCAs obtained for the women studied using both Miraluma® and B.E.S.T. imaging. The MCAs were almost identical for those with normal breast tissue, suggesting that the enhanced approach does not alter the delivery or uptake of sestamibi in normal breast tissue. Individuals with inflammatory changes of the breast, however, showed statistically significant differences in MCA, which were enhanced by B.E.S.T. imaging. These differences were even greater for individuals with breast cancer. These mean±standard deviations MCAs are shown in Table 1 and are statistically significant.

TABLE 1

| | | Inflammatory | |
|---|---|---|---|
| | Normal | Changes | Cancer |
| Miraluma ® | ˜107.5 ± 21.9 | ˜184.0 ± 19.2 | ˜282.5 ± 14.8 |
| B.E.S.T. | ˜125.5 ± 31.5 | ˜228.8 ± 24.0 | ˜442.0 ± 5.7 |
| p-level | Not Significant | P < 0.05 | P < 0.005 |

The results shown in Table 1 and FIG. 3 show no difference between results obtained using either the resting Miraluma® (M) approach or the B.E.S.T. (B) approach in normal patients. The MCA using Miraluma® was about 107.5±21.9 and was almost identical to that seen (about 125.5±31.5) with B.E.S.T.

In women who had inflammatory changes, Miraluma® had a statistically lower (p<0.05) MCA of about 184.0±19.2 compared with that seen with B.E.S.T. imaging (about 228.8±24.0). The differences were more significant (p<0.005) for patients with breast cancer, where B.E.S.T. imaging had a MCA of about 442.0±5.7 and Miraluma®had a MCA of about 282.5±14.8.

This study demonstrated that breast cancer has increased vascularity which can be affected by the vasodilatory effects of dipyridamole, and greater mitochondrial activity which can be further detected once increased delivery of isotope is provided through enhanced blood flow.

### Study 2: Results

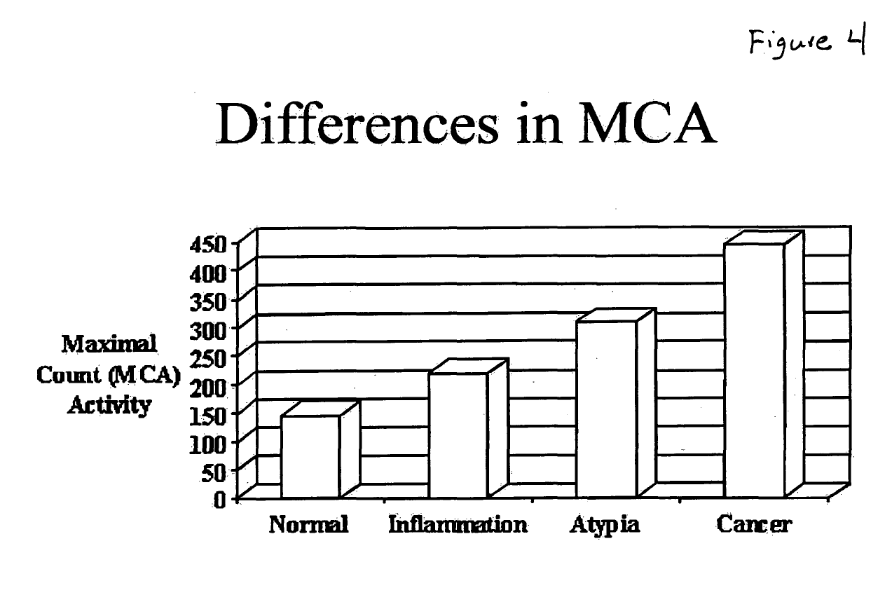

In the second part of the study, the outcomes of histopathology and MCA results using B.E.S.T. imaging were compared. FIG. 4 shows a graphic representation of the results of MCA as seen in normal breast tissue, inflammatory tissue, and breast cancer tissue. MCA is plotted and displayed showing a grouping of results revealing differences between normal tissues, inflammatory tissues, and breast cancer. These differences displayed an exponential increase in MCA progressing from “normal” to “cancer.”

The results shown in FIG. 4 reveal an exponential increase in MCA proceeding from “normal” to “inflammatory” to “cancer”. The MCA of patients with normal breast tissue (n=88) ranged from about 80 to about 202 with an average value of about 145.0±29.1. The 95% CI for “normal” breast tissue was about 139 to about 151. Individuals with inflammatory changes (n=77) had MCAs ranging from about 130 to about 298 with an average of about 218.0±40.3, with a 95% CI of 209 to 227. There were 15 individuals with cellular atypia whose MCAs ranged from about 209 to about 333 with an average value of about 307.7±29.3. The 95% CI for patients with atypia was about 292 to about 323. Patients with breast cancer (n=15) had a 95% CI of 399 to 491 with an average MCA of about 445.3±83.3 and a range in values from about 270 to about 594. Breast cancers in this study ranged from about 4 mm to about 2 cm with the average size being about 8 to about 10 mm.

When analyzed for differences between groups, there was a statistically significant difference between normal and inflammatory tissue, inflammatory and atypia tissue, and between atypia and cancerous tissue. In each instance, the increase was statistically significant at the p<0.001 level. The raw data (FIG. 4) were analyzed following histopathologic results. The mean MCAs for each of four groups (normal, inflammatory, atypia and cancer) are displayed. These mean+standard deviation MCAs are shown in Table 2 and are statistically significant.

TABLE 2

| | | Inflammatory | Atypia/ | |
|---|---|---|---|---|
| | Normal | Changes | Metaplasia | Cancer |
| Maximal | ˜145.0 ± | ˜218.0 ± | ˜307.7 ± | ˜445.3 ± |
| Count | 29.1 | 40.3 | 29.3 | 83.3 |
| Activity | | | | |
| (MCA) | | | | |

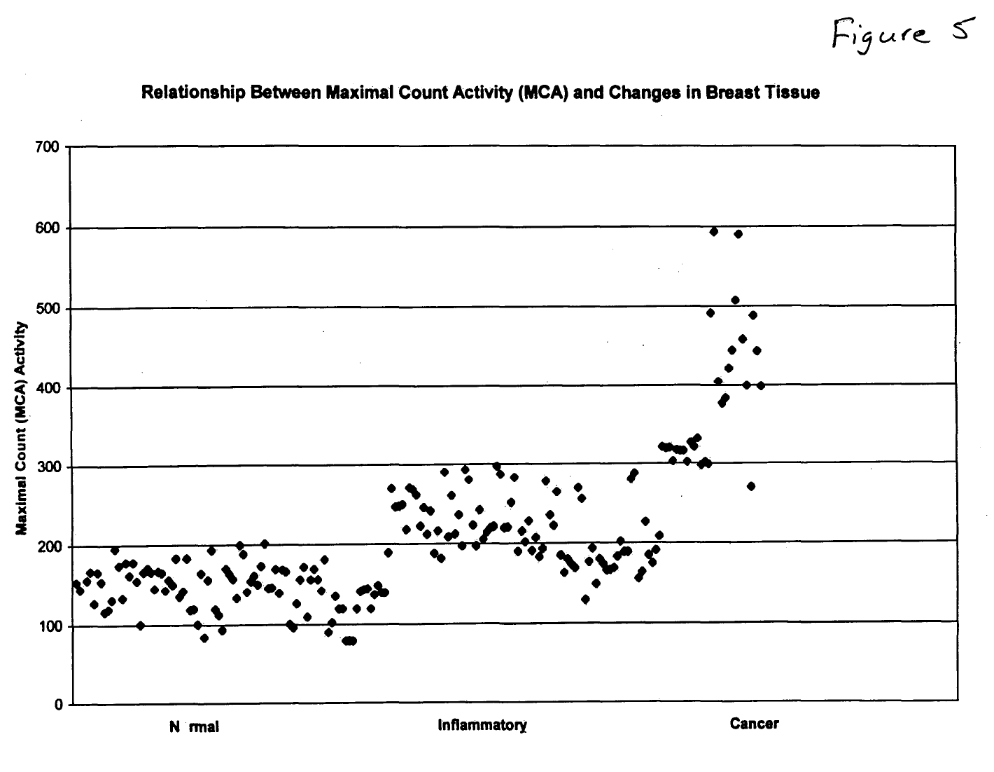

FIG. 5 shows differences in maximal count activity in individuals with normal, inflammatory, atypia and cancerous breast tissue. FIG. 5 shows the bar graph comparisons for the four groups. The results demonstrate overlap between normal and inflammatory and between inflammatory and atypia, suggesting a possible transition from stage to stage. The overall appearance of cancer differed from the appearance of pre-cancers (atypia, hyperplasia, etc.). Breast cancer appeared more circular consistent with a “mass effect” while atypia, hyperplasia and DCIS have values intermediate between inflammatory changes and cancer. These findings are consistent with increased mitochondrial activity present in these cells without increased vascularity. The appearance of DCIS is typically tubular, following the ducts.

One cancer with a MCA of 270 clearly fell within the MCA range for inflammatory tissue. On further inspection, the lymph nodes were negative and the tumor had little evidence of angiogenesis. It was surgically removed with no additional radiation therapy or chemotherapy recommended to the patient by her oncology team.

The findings of this study demonstrate differences based upon vascularity and mitochondrial activity and demonstrate the ability to distinguish across a continuum of breast tissue changes ranging from normal to inflammatory to atypia to cancer. These distinctions support a transition from normal breast tissue to breast “cancer” and offer a method for distinguishing between changes in breast tissue and earlier detection and monitoring of breast cancer.

It should be understood that various changes and modifications to the preferred embodiment described herein would be apparent to those skilled in the art. While the present invention has been tested and is described in connection with the detection of breast cancer, the principles of the present invention are equally applicable to so-called “hard tumors” (pre-cancerous, and cancerous) of other origins or locations. For example, the present invention can be used in the detection of thymus abnormalities and coronary hearth disease where inflammation is present. Such changes and modifications can be made without departing from the spirit and scope of the present invention and without diminishing its attendant advantages. It is therefore intended that such changes and modifications be covered by the appended claims.

What is claimed is:

1. A method of detecting abnormal tissue in a patient comprising:
(a) administering to the patient a vasodilatory agent capable of increasing the uptake of a radiopharmaceutical which selectively accumulates in abnormal tissue;
(b) administering to the patient a radiopharmaceutical agent which selectively accumulates in the abnormal tissue; and
(c) imaging a tissue of the patient with a radiation detector to detect any abnormal tissue.

2. The method of claim 1 wherein the abnormal tissue is abnormal breast tissue.

3. The method of claim 1 wherein the vasodilatory agent comprises dipyridamole.

4. The method of claim 3, further comprising administering the dipyridamole in a dose of between about 30 mg and about 120 mg.

5. The method of claim 3 further comprising administering the dipyridamole intravenously in a dose of between about 30 cc and about 60 cc.

6. The method of claim 3 further comprising administering 0.852 milligram per kilogram (mg/kg) of body weight of dipyridamole intravenously.

7. The method of claim 1 wherein the radiopharmaceutical is a sestamibi.

8. The method of claim 7 wherein the sestamibi is Technetium-99m hexakis 2-methoxyisobutylisonitrile.

9. The method of claim 7 further comprising administering sestamibi in a dose of between about 20 mCi and about 35 mCi.

10. The method of claim 9 further comprising administering the sestamibi intravenously in a dose of between about 25 mCi and about 30 mCi.

11. The method of claim 1 wherein the abnormal tissue comprises cancerous tissue.

12. The method of claim 1 wherein the abnormal tissue comprises inflammatory tissue.

13. The method of claim 1 wherein the abnormal tissue comprises atypia tissue.

14. The method of claim 1 wherein the radiation detector is selected from the group consisting of single photon emission computed tomography (SPECT) detectors, positron emission tomography (PET) detectors, semiconductor detectors, and other suitable planar imaging devices.

15. The method of claim 1 further comprising administering the vasodilatory agent and the radiopharmaceutical intravenously.

16. The method of claim 1 further comprising administering the radiopharmaceutical during the peak vasodilatory effect of the vasodilatory agent.

17. A method for early detection of abnormal tissue comprising measuring vascularity activity and mitochondrial activity.

18. The method of claim 17 wherein the abnormal tissue is abnormal breast tissue.

19. The method of claim 17 further further comprising increasing the vascularity activity by a vasodilatory effect of dipyridamole.

20. The method of claim 19 further comprising administering the dipyridamole in a dose of between about 30 mg and about 1 20 mg.

21. The method of claim 19 further comprising administering the dipyridamole intravenously in a dose of between about 30 cc and about 60 cc.

22. The method of claim 19 further comprising administering about 0.852 milligram per kilogram (mg/kg) of body weight of dipyridamole intravenously.

23. The method of claim 17 further comprising increasing mitochondrial activity through the increased delivery of a sestamibi.

24. The method of claim 23 wherein the sestamibi is Technetium-99m hexakis 2-methoxyisobutylisonitrile.

25. The method of claim 24 further comprising administering the sestamibi in a dose of between about 20 mCi and about 35 mCi.

26. The method of claim 24 further comprising administering the sestamibi intravenously in a dose of between about 25 mCi and about 30 mCi.

27. The method of claim 17 wherein the abnormal tissue comprises cancerous tissue.

28. The method of claim 17 wherein the abnormal tissue comprises inflammatory tissue.

29. The method of claim 17 wherein the abnormal tissue comprises atypia tissue.

30. The method of claim 17 further comprising imaging a tissue of the patient with a radiation detector to detect any abnormal tissue.

31. The method of claim 30 wherein the radiation detector is selected from the group consisting of single photon emission computed tomography (SPECT) detectors, positron emission tomography (PET) detectors, semiconductor detectors, and other suitable planar imaging devices.

32. The method of claim 17 further comprising administering the vasodilatory agent and the radiopharmaceutical intravenously.

33. The method of claim 17 further comprising administering the radiopharmaceutical during a peak vasodilatory effect of the vasodilatory agent.

34. A method for detecting changes in tissue comprising measuring increased vascularity activity and increased mitochondrial activity.

35. The method of claim 34 wherein the tissue comprises breast tissue.

36. The method of claim 34 further comprising increasing the vascularity activity by a vasodilatory effect of a dipyridamole.

37. The method of claim 36 further comprising administering the dipyridamole in a dose of between about 30 mg and about 120 mg.

38. The method of claim 36 further comprising administering the dipyridamole intravenously in a dose of between about 30 cc and about 60 cc.

39. The method of claim 36 further comprising administering about 0.852 milligram per kilogram (mg/kg) of body weight of the dipyridamole intravenously.

40. The method of claim 34 further comprising increasing mitochondrial activity through the increased delivery of a sestamibi.

41. The method of claim 40 wherein the sestamibi is Technetium-99m hexakis 2-methoxyisobutylisonitrile.

42. The method of claim 41 further comprising administering the sestamibi in a dose of between about 20 mCi and about 35 mCi.

43. The method of claim 41 further comprising administering the sestamibi intravenously in a dose of between about 20 mCi and about 35 mCi.

44. The method of claim 34 wherein the tissue is an abnormal tissue comprising cancerous tissue.

45. The method of claim 34 wherein the tissue is an abnormal tissue comprising inflammatory tissue.

46. The method of claim 34 the tissue is an abnormal tissue comprising atypia tissue.

47. The method of claim 34 further further comprising imaging the tissue of the patient with a radiation detector to detect any abnormal tissue.

48. The method of claim 47 wherein the radiation detector is selected from the group consisting of single photon emission computed tomography (SPECT) detectors, positron emission tomography (PET) detectors, semiconductor detectors, and other suitable planar imaging devices.

49. The method of claim 34 further comprising administering a vasodilatory agent and a radiopharmaceutical intravenously.

50. The method of claim 34 further comprising administering a radiopharmaceutical during a peak vasodilatory effect of a vasodilatory agent.

51. A formulation for early detection of cancer comprising combining a dipyridamole and a sestamibi.

52. The formulation of claim 51 further comprising about 0.852 milligram per kilogram (mg/kg) of body weight of the dipyridamole and about 20-35 mCi of the sestamibi.

53. The formulation of claim 51 further comprising about 0.852 milligram per kilogram (mg/kg) of body weight of the dipyridamole and about 25-30 mCi of the sestamibi.

54. A method of detecting abnormal tissue in a patient comprising:
(a) administering to the patient a biologically effective dose of a dipyridamole agent;
(b) administering to the patient a biologically effective dose of a sestamibi; and
(c) imaging at a tissue of the patient with a radiation detector to detect abnormal tissue in the patient.

55. The method of claim 54 wherein the abnormal tissue comprises abnormal breast tissue.

56. The method of claim 54 wherein the biologically effective dose of the dipyridamole agent is a dose of between about 30 mg and about 120 mg.

57. The method of claim 54 wherein the biologically effective dose of the dipyridamole agent is a dose of between about 30 cc and about 60 cc.

58. The method of claim 54 wherein the biologically effective dose of the dipyridamole agent is a dose of about 0.852 milligram per kilogram (mg/kg) of body weight.

59. The method of claim 54 wherein the biologically effective dose of the sestamibi is a dose of between about 20 mCi and about 35 mCi.

60. The method of claim 59 further comprising administering the sestamibi intravenously in a dose of between about 25 mCi and about 30 mCi.

61. The method of claim 54 wherein the abnormal tissue comprises cancerous tissue.

62. The method of claim 54 wherein the abnormal tissue comprises inflammatory tissue.

63. The method of claim 54 wherein the abnormal tissue comprises atypia tissue.

64. The method of claim 54 wherein the radiation detector is selected from the group consisting of single photon emission computed tomography (SPECT) detectors, positron emission tomography (PET) detectors, semiconductor detectors, and other suitable planar imaging devices.

65. The method of claim 54 further comprising administering the dipyridamole agent and the sestamibi intravenously.

66. The method of claim 54 further comprising administering the sestamibi during a peak vasodilatory effect of the dipyridamole agent.
